(19) United States
(12) Patent Application Publication
(10) Pub. No.: US 2017/0231220 A1
(43) Pub. Date: Aug. 17, 2017

(54) ANTIMICROBIAL COMPOSITION

(71) Applicant: Henkel IP & Holding GmbH, Duesseldorf (DE)

(72) Inventors: Jessica Lawshe, Phoenix, AZ (US); Janice L. Fuls, Fountain Hills, AZ (US); Gregory Cole, Phoenix, AZ (US); Nancy D. Rodgers, Chandler, AZ (US)

(73) Assignee: Henkel IP & Holding GmbH, Duesseldorf (DE)

(21) Appl. No.: 15/586,030

(22) Filed: May 3, 2017

Related U.S. Application Data
(63) Continuation-in-part of application No. 14/089,091, filed on Nov. 25, 2013.

(51) Int. Cl. A01N 33/12; A61Q 17/00; A61K 8/27; A01N 59/16; A61K 8/41

(52) U.S. Cl. CPC A01N 33/12; A01N 59/16; A61K 8/416; A61K 8/27; A61Q 17/005; A61K 2800/74; A61K 2800/10

(57) ABSTRACT

An anti-microbial composition including: a solvent; a cationic active ingredient; a source of zinc ions, wherein the zinc ions have a concentration from 6.2×10⁻⁴ to 8.7×10⁻³ mol/L; and an amine oxide surfactant. A method of selectively killing Gram-positive bacteria, the method including: exposing bacteria to a formulation including: 0.05 to 0.3 wt. % of benzethonium chloride and/or benzalkonium chloride; a source of zinc ions, wherein the zinc ions have a concentration from 6.8×10⁻³ to 2×10⁻² mol/L; and an amine oxide surfactant.

### FIELD OF THE INVENTION

The present invention generally relates to a composition with bactericidal activity against both Gram-positive and Gram-negative bacteria. The present invention also discloses a composition useful to selectively kill Gram-positive bacteria in the presence of Gram-negative bacteria.

### BACKGROUND

In any given day an individual may encounter multiple instances of bacterial life or other microorganisms, some of which may be harmful to the person. For example, raw meat may be a breeding ground for bacteria which, if ingested, may cause illness. Antibacterial agents are personal care products that are used to inhibit bacteria growth and/or kill bacteria. For example, an antimicrobial soap may be used to wash skin that has come in contact with bacteria to neutralize the bacteria's harmful effects.

Accordingly, it is desirable to have an antimicrobial composition that has high efficacy in killing bacteria, or inhibiting bacteria growth when such an antimicrobial composition is used on the skin. Additionally, it is desirable to have an antimicrobial composition that is safe for the environment and consumer use and that is compatible with other soap ingredients that deliver a satisfactory consumer experience. Furthermore, other desirable features and characteristics of the present examples will become apparent from the subsequent detailed description and the appended claims, taken in conjunction with the accompanying drawings and this background.

### BRIEF SUMMARY

Accordingly, this disclosure discusses, an antimicrobial composition, comprising: a solvent; a cationic active ingredient; a source of zinc ions, wherein the zinc ions have a concentration from 6.2×10⁻⁴ to 8.7×10⁻³ mol/L; and an amine oxide surfactant.

This disclosure also discusses an antimicrobial product exhibiting increased efficacy, including: a container and an antimicrobial composition housed within the container, wherein the antimicrobial composition includes: a quaternary ammonium compound, wherein at least one substitution on the amine comprises a aromatic ring, the quaternary ammonium compound comprising 0.05 to 0.30 wt. % of the composition; a zinc salt, wherein the zinc ions have a concentration from 1.5×10⁻³ to 7.7×10⁻³ mol/L; an amine oxide surfactant, comprising 0.9 to 1.9 wt. % of the antimicrobial composition; cetrimonium chloride; and a polar solvent.

Among other examples, this disclosure discusses, a method of selectively killing Gram-positive bacteria, the method including: exposing bacteria to a formulation including: 0.05 to 0.3 wt. % of benzethonium chloride and/or benzalkonium chloride; a source of zinc ions, wherein the zinc ions have a concentration from 6.8×10⁻³ to 2×10⁻² mol/L; and an amine oxide surfactant.

### BRIEF DESCRIPTION OF THE DRAWINGS

The present disclosure includes the following drawing figures, wherein like numerals denote similar, but not necessarily identical, elements, and

FIG. 1 is a diagram of an example of applying an antimicrobial cleaning product to skin according to the principles described herein.

FIG. 2 is a flowchart of a method of selectively killing Gram-positive bacteria according the principles described herein.

### DETAILED DESCRIPTION

The following detailed description of the invention is merely exemplary in nature and is not intended to limit the invention or the application and uses of the invention. Furthermore, there is no intention to be bound by any theory presented in the disclosure.

As described above, individuals come into contact with bacteria and other microorganisms on a day-to-day basis. Many of these bacterial compounds and other microorganisms may be harmful to humans. In one example, humans may come into contact with the bacteria via their skin. Bacteria on the skin can be harmful, and can also facilitate the ingestion of the bacteria, which may pose additional health risks. For example, a human preparing a meal may get bacteria on their hands from raw meat. As the fingers come into contact with the mouth, the bacteria may be ingested, and cause additional health problems for an individual. Accordingly, antimicrobial soaps are used to kill the bacteria that come into contact with skin, and to prevent further health problems for individuals who come in contact with the bacteria either directly, or indirectly.

While antimicrobial cleaning products may be beneficial, some antimicrobial cleaning products may exhibit less than satisfactory characteristics. For example, many antimicrobial active ingredients may be harmful to the environment and the individual. More specifically, some antimicrobial active ingredients, such as Triclosan have been criticized as being possible environmental toxins, endocrine disrupters, toxins, and contributors to bacterial resistance. Other active ingredients, such as benzethonium chloride may also present other inefficiencies. For example, some antimicrobial soap formulations that include cationic active components such as benzethonium chloride may exhibit efficacy in in vitro type methods, yet may experience a reduction in efficacy when used on skin, or may be inactive when used on the skin. This may be due to the positively-charged cationic antimicrobial ingredients which may be neutralized by other ingredients found in cleansing formulas. The positively-charged cationic antimicrobial ingredient may be neutralized by negatively-charged skin cells and/or bacteria.

Accordingly, the principles described herein provide a composition for increasing the on-skin efficacy of an antimicrobial cleaning product that would otherwise exhibit a reduced ability to kill bacteria, or reduce bacteria growth as described above. More specifically, the principles described herein provide a formulation in which high antimicrobial efficacy is exhibited. Such a composition may include a cationic active ingredient to kill bacteria, reduce bacteria growth, or combinations thereof. Moreover, the composition may also include a surfactant. The composition of this form may be beneficial in that it uses an environmentally-friendly and consumer-safe active ingredient while maintaining an ability to kill bacteria and reduce bacteria growth without negatively impacting other cleaning product ingredients.

Further, not all antimicrobial agents are equally effective on all types of microbes. For example, bacteria are often divided into Gram-negative and Gram-positive groups based on their reaction with a stain developed by Hans Gram in 1884. Gram-positive bacterial are colored violet by exposure to Gram stain. The difference in the cell walls between the Gram-positive and Gram-negative bacteria may produce different responses between the two groups to different anti-microbial agents. Hazardous microbes are found in both the Gram-positive and Gram-negative groups. Accordingly, it is desirable for antimicrobial products to inhibit or kill both Gram-positive and Gram-negative microbes in order to provide broad antimicrobial functionality.

Accordingly, the antimicrobial cleaning product is safe for consumers, safe for the environment, effectively kills both Gram-positive and Gram-negative bacteria on the skin, and maintains properties that give the cleaning product good performance qualities and good aesthetic qualities.

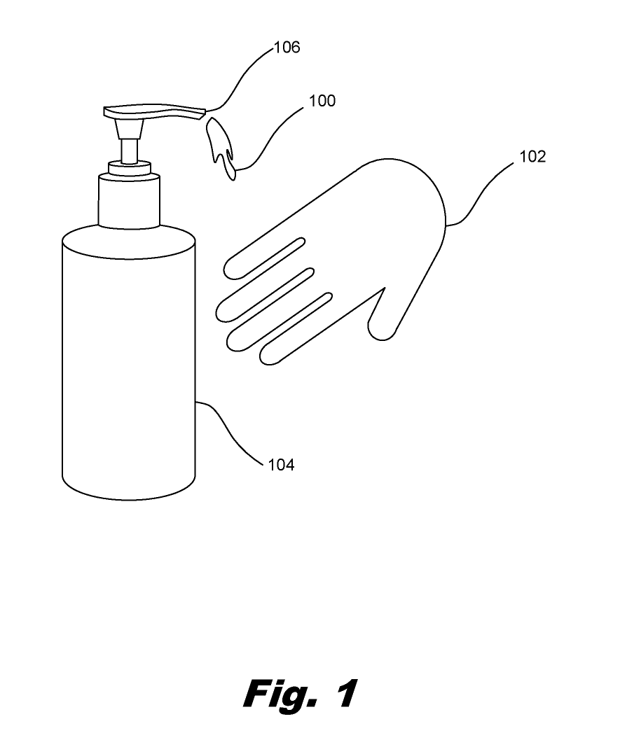

Turning now to the figures, FIG. 1 is a diagram of an example of applying an antimicrobial cleaning product (100) to skin (102) according to the principles described herein. As depicted in FIG. 1, the antimicrobial cleaning product (100) may be applied to a hand. However, the antimicrobial cleaning product (100) may also be applied on other skin surfaces such as the arms and face of an individual. As described above the antimicrobial cleaning product (100) may exhibit increased efficacy when used on the skin. As used in the present specification, and in the appended claims, efficacy may refer to the ability of an antimicrobial cleaning product (100) to reduce bacteria growth, or to kill bacteria. The efficacy of an antimicrobial cleaning product (100) may be measured using a logarithmic reduction scale or as a percent reduction in a bacteria population. For example, a 1 log reduction corresponds to a 90% reduction in the bacteria population. Similarly, a 2 log reduction corresponds to a 99% reduction in the bacteria population, a 3 log reduction corresponds to a 99.9% reduction in the bacteria population, a 4 log reduction corresponds to a 99.99% reduction in the bacteria population, and a 5 log reduction corresponds to a 99.999% reduction in the bacteria population.

In some examples, the antimicrobial cleaning product (100) may be held in a container (104) that has an opening (106) that allows the cleaning product (100) to flow out of the container (104) onto the skin (102). In one example, the container (104) is a pump-type container (104) that expels the cleaning product (100). For example, a user may depress a handle on the pump-type container (104) which actuates a pump that draws liquid up a siphon tube and expels the antimicrobial cleaning product (100) out of a nozzle opening (106) of the pump-type container (104).

The antimicrobial cleaning product (100) may include an antimicrobial composition. The antimicrobial composition may include an active ingredient that kills bacteria and other microorganisms that come into contact with the skin. The antimicrobial composition may also include a mechanism for delivering the antimicrobial composition onto the skin cells to interact with the bacteria cells. The antimicrobial composition may also include other ingredients to increase the “feel” and aesthetic qualities of the antimicrobial cleaning product. Such ingredients may include a dye, a fragrance, a preservative, a pH adjuster, an antiseptic, a humectant, skin conditioners, a thickener, a conditioning agent, a chelating agent, a viscosity adjuster, and/or a foam booster among other ingredients. Specific examples of ingredients that may be used in the antimicrobial composition are given in more detail below. In some examples, the antimicrobial composition may be a gel-type composition. In this example, the composition may have a pH between 4.5 and 5.3. In other examples, the antimicrobial composition may be a foam-type composition. In this example, the foam-type composition may have a pH between approximately 5 and 6. The antibacterial cleaning product (100) may also include a polar carrier, such as water, to contain the antimicrobial cleaning product (100) ingredients. A more detailed description of the different ingredients in the antimicrobial composition is given as follows.

Previous work, described in the U.S. Ser. No. 14/089,091 application, showed a variety of antimicrobial formulations using cationic active ingredients to produce log 2 or greater reductions in bacteria. Further work has revealed an unexpected interaction between the concentration of zinc ions and antimicrobial activity. Generally speaking, the effectiveness of an active ingredient depends on its availability in the formulation. This effectiveness increases with concentration of the active ingredient. It is common for the effectiveness to asymptotically approach a maximum as the free concentration increases. Similarly, it is common to see a linear response at low concentrations. Accordingly, range testing can be used to determine where on such a curve the concentration of the active ingredient is and then make adjustments. For example, the concentration of the active ingredient may be increased to at least the point where the behavior becomes non-linear, indicating a decreasing incremental benefit to higher concentrations of the active ingredient.

While studying the effectiveness of antimicrobial formulations containing zinc and quaternary amines, the accumulating data did not show a normal response pattern. For example, Experimental Example 1, below, used a set of formulations where the zinc level was varied and the other components of the formulation were kept constant.

The Chlorine Equivalency test (AOAC 955.16) is an in vitro method used to evaluate a product's ability to kill Staphylococcus aureus and Salmonella typhi. The test uses three chlorine standards: 200 ppm, 100 ppm, and 50 ppm to provide comparative performance. The product and standards are inoculated with a population of test bacteria ten times every 1.5 minutes. One minute after each inoculation, a portion of the test material is transferred to a subculture tube. Growth in the product's subculture tubes are compared to the subculture tubes of the chlorine standards to determine at which concentration of chlorine the product is equivalent to. A pass result is equivalent to or better than the 50 ppm chlorine control. A fail result is worse than the 50 ppm chlorine control.

In this example the tested formulations were constant except for the varying levels of zinc sulfate. The test formulations included: a varying amount of zinc sulfate (ZnSO₄), 0.13 wt. % benzalkonium chloride (BAC), 1.5 wt. % cetrimonium chloride (CC), and 0.2 wt. % chlorohexidine gluconate (CHG). Previous studies with the higher level of zinc sulfate had shown a loss of efficacy vs. Salmonella. This dose study shows that as the level of zinc sulfate increases efficacy is negatively impacted against this Gram-negative organism. However, the formulations with 0.15 wt. % or more ZnSO₄ remained effective against Staphylococcus aureus which is Gram-positive. And the 0.05 to 0.10 wt. % zinc sulfate formulations were effective against both test organisms. Subsequent testing has shown that similar anti-bacterial activity can be achieved with formulations that did not include chlorohexidine gluconate.

### EXPERIMENTAL EXAMPLE 1

| | Staphylococcus | Salmonella |
|---|---|---|
| | aureus | typhi |
| 0.05 wt. % ZnSO₄ | Pass | Pass |
| 0.10 wt. % ZnSO₄ | Pass | Pass |
| 0.15 wt. % ZnSO₄ | Pass | FAIL |
| 0.20 wt. % ZnSO₄ | Pass | FAIL |

This observation of reduced effectiveness with higher zinc concentrations was also found in a standardized time kill experiment. This study agrees with the observation in the chlorine equivalency study that at higher concentrations of zinc sulfate, efficacy vs. Gram-negative bacteria is lost. In this example, seven Gram-negative organisms (identified by name and American Type Culture Collection number) were tested against two formulas containing 0.10 wt. % and 0.25 wt. % zinc sulfate respectively. The results are reported in log 10 reductions of surviving organisms, i.e. a value of 2 represents 1% survival. Results <1 log reduction are considered to show low or no activity; results of 1 log-2 log reduction indicate moderate activity; and results of 3 log reduction or greater indicate high activity.

TABLE 2

| | | 1 | | 2 | |
|---|---|---|---|---|---|
| | | BAC 0.13 wt. % | | BAC 0.13 wt. % | |
| | | with 0.10 wt. % | | with 0.25 wt. % | |
| | | Zinc Sulfate | | Zinc Sulfate | |
| Organism | ATCC | 15 sec. | 30 sec. | 15 sec. | 30 sec. |
| K. pneumoniae | 10031 | 4.38 | >6.20 | 0.48 | 1.10 |
| E. coli | 11229 | 4.40 | 4.80 | 0.05 | 0.19 |
| S. enterica | 13076 | 5.77 | >6.30 | 0.20 | 0.92 |
| S. sonnei | 11060 | 5.11 | >5.85 | 0.26 | 1.14 |
| P. aeruginosa | 9027 | 4.15 | >4.88 | 0.72 | 1.17 |
| S. flexneri | 700930 | >5.78 | >5.78 | 0.48 | 0.83 |
| E. coli (O157:H7) | 43888 | >5.84 | >5.84 | 0.06 | 1.02 |

The formula with the 0.25 wt. % zinc sulfate (ZnSO₄) showed minimal germ kill at either 15 or 30 seconds. The 0.25 wt. % zinc sulfate formulation, which included other non-zinc active components, reached only reached 1 log reduction at 30 seconds for four of seven test organisms. In contrast, the formula with the 0.10 wt. % zinc sulfate showed high rates of effectiveness with over 4 log reductions at 15 seconds and ˜5 log reductions at 30 seconds. The large difference in germ kill between the low concentration zinc formulation and the high concentration zinc formulation were surprising as generally higher levels of active ingredients, like zinc, are associated with higher rates of germ kill.

These results suggest two conclusions: first, a highly effective antimicrobial composition can be made using a moderate amount of zinc combined with a quaternary ammonium chloride, this composition is capable of achieving four to five log reductions in both Gram-positive and Gram-negative bacteria in 15 to 30 seconds of exposure; and second, the use of similar formulations with higher concentration zinc may be used to selective killing Gram-positive bacteria while retaining Gram-negative species. The first composition therefore provides an effective antimicrobial composition that is effect against Gram-positive and Gram-negative species. As shown in table 2, even relatively short contact times of 15 second produce four or more orders of magnitude reduction in living bacteria. That is, less than 0.01% of the exposed bacteria remain viable at 15 seconds. In contrast, commercially available products often achieve 1 or 2 orders of magnitude reduction. Data from testing five commercially available products is available in the U.S. Ser. No. 14/089,091 application. That testing, using the Health Care personnel Hand Wash method, showed a 0.047 to 1.7 log10 reduction in S. marcescens bacteria.

The second composition may have value in rapidly and efficiently killing Gram-positive bacteria from a mixed culture. This may be useful to culture only Gram-negative species. This may reduce the need to Gram stain the slides. This may be a tool to facilitate rapid identification of bacteria in a mixed culture. While such selectivity is less useful in a general cleaning formulation, there exist a variety of scientific uses for a safe and efficient means of selectively killing one type or category of bacteria in a mixed culture.

In one example, an antimicrobial formulation (Formulation 1) comprises the following components:

### Formulation 1

| | | Example wt. % |
|---|---|---|
| Component | Example Species | of formulation |
| Quaternary | Benzalkonium Chloride, | 0.11 to 0.26 |
| Ammonium | Benzethonium Chloride | |
| Chloride | | |
| Zinc Ion Source | ZnSO₄, Zinc Gluconate, ZnCl₂ | 0.05 to 0.10 |
| Surfactant | Lauryldimethylamine oxide, | 0.1 to 3 |
| | Tetradecyldimethylamine oxide | |
| Solvent | Water, Ethanol | Q.S. |

The following formulation is an example of a commercial formulation using the principles described herein.

### Formulation 2

| | Weight |
|---|---|
| Component | Percentage (wt. %) |
| Benzalkonium Chloride and/or Benzethonium | 0.1 to 0.3 |
| Chloride | |
| Viscosity Modifier | 0.1 to 1 |
| Chelating Agent | 0.01 to 0.05 |
| Nonionic Surfactant | 1 to 2 |
| Humectant | 0.5 to 2 |
| Citric Acid | 0.05 to 0.4 |
| Sunflowerseedamidopropyl Ethyldimonium | 0.4 to 1.2 |
| Ethosulfate and PEG-90 | |
| Cetrimonium Chloride | 0.5 to 3 |
| Amphoteric Surfactant | 0.1 to 1 |
| Preservative | 0.1 to 0.5 |
| Zinc Sulfate | 0.05 to 0.1 |
| Fragrance | 0.2 to 0.25 |
| Dyes | 0.00001 to 0.000004 |
| Water | Q.S. |

The antimicrobial composition includes a cationic active ingredient. The cationic active ingredient may be any positively charged ingredient that is used to kill bacteria or other microorganisms that come in contact with skin surfaces. For example, the cationic active ingredient may be benzethonium chloride. In another example, the cationic active ingredient may be benzalkonium chloride. The cationic active ingredient may be a combination of benzethonium chloride and benzalkonium chloride. The cationic active ingredient may comprise between 0.10 weight percent and 0.20 weight percent of the antimicrobial composition. Including a cationic active ingredient may be beneficial as such ingredients are relatively environmentally-friendly and safe for consumer use.

The antimicrobial composition may include an amine oxide. In some examples, the amine oxide includes two small alkyl groups and one longer akyl group attached to the nitrogen group. The small alkyl groups may be linear or branched C₁ to C₃ alkyl groups. The amine oxide may be 0.5 to 3 wt. % of the antimicrobial composition. The amine oxide may be lauramine oxide, with two methyl groups and a C₁₂ group. The lauramine oxide surfactant may enhance the deposition of the active ingredient, such as the benzethonium chloride on the skin. In some examples, the lauramine oxide may comprise between 0.9 weight percent to 1.9 weight percent of the antimicrobial composition. More specifically, the lauramine oxide surfactant may comprise 1.50 weight percent of the antimicrobial composition. The lauramine oxide surfactant may assist in killing bacteria by lowering the surface tension of a liquid or the interfacial tension between two liquids or between a liquid and a solid. When added to the antimicrobial composition, the lauramine oxide surfactant reduces the surface tension of the antimicrobial composition allowing the composition to penetrate the skin (102) rather than slide off the skin (102). The result is that the antimicrobial composition can function more effectively.

The antimicrobial composition may include a number of co-surfactants. A co-surfactant may enhance the ability of the amine oxide surfactant to reduce the surface tension of the antimicrobial composition. In some examples, the co-surfactant may be a zwitterionic surfactant. Examples of co-surfactants include: cocamidopropyl betaine, sunfloweramidopropyl ethonium sulfate, PEG-120 methyl glucose dioleate, lauryl/myristyl amidopropyl amine oxide, lauramidopropylamine oxide, or combinations thereof.

The antimicrobial composition may also include a thickener to increase the “feel” of the antimicrobial cleaning product (100). In one example, the thickener is a water soluble polymer. The thickener may include hydromethylcellulose, PEG-120 methyl glucose, cocamide MEA, hydroxypropyl methylcellulose, or combinations thereof. In some examples, the antimicrobial cleaning product (100) may be Lauramide DEA free. Salts, e.g., sodium chloride, and hydrophilic species may be used to modify the viscosity of the antimicrobial composition.

In some examples, the antimicrobial composition may include a zinc salt. The zinc salt may be an organic salt. The zinc salt may be an inorganic salt. Specific examples of zinc salts that may be included in the antimicrobial composition include zinc sulfate, zinc gluconate, zinc chloride, or combinations thereof. However, a wide variety of zinc salts may be used to provide the zinc ions without reducing the effectiveness of the formulation.

As indicated above, the antimicrobial composition and the antimicrobial cleaning product (100) described above may be beneficial in that an environmentally-friendly and consumer safe active ingredient is used, while maintaining the efficacy of the active ingredient. Maintaining the efficacy of the active ingredient may include using ingredients in the antimicrobial cleaning product (100) that reduces the neutralization of the active ingredient. Moreover, the antimicrobial cleaning product (100) as described herein is compatible with other ingredients that give the antimicrobial cleaning product (100) good performance characteristics and good aesthetic qualities. These benefits, in addition to others, are provided by the antimicrobial composition as described herein.

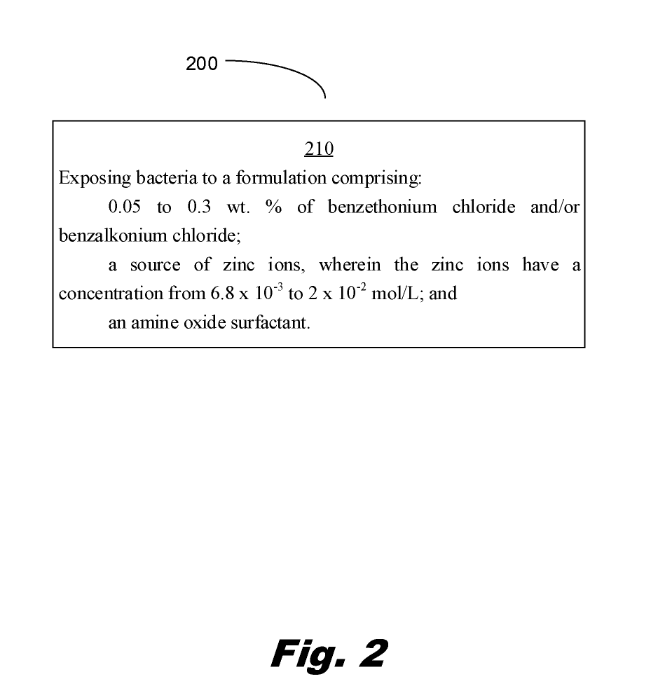

In FIG. 2, a method (200) is disclosed for selectively reducing the Gram-positive organisms in a mixture of bacteria. The mixture is subjected to a formulation of a quaternary ammonium chloride (e.g., BAC) with a higher zinc concentration such that the Gram-positive bacteria are killed while the Gram-negative bacteria are not. In one example the method comprises: (210) applying a formulation to a mixture of bacteria, wherein the formulation comprises: 0.05 to 0.3 wt. % of benzethonium chloride and/or benzalkonium chloride; a source of zinc ions, wherein the zinc ions have a concentration from 6.8×10⁻³ to 2×10⁻² mol/L; and an amine oxide surfactant.

The method may include additional steps. For example, the method may further comprise rinsing the formulation from the bacteria after a designated period of time. In one example, the time is from 5 sec to 120 seconds. The method may further comprise plating the bacteria onto a media. This may facilitate exposing the bacteria and also rinsing the formulation from the bacteria. In another example, the formulation may be a growth media, for example, to provide additional selectivity while allowing the bacteria to multiply.

While at least one example has been presented in the foregoing detailed description of the invention, it should be appreciated that a vast number of variations exist. It should also be appreciated that the described examples are only examples, and are not intended to limit the scope, applicability, or configuration of the disclosed subject matter in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antimicrobial composition, comprising:
a solvent;
a cationic active ingredient;
a source of zinc ions, wherein the zinc ions have a concentration from 6.2×10⁻⁴ to 8.7×10⁻³ mol/L; and
an amine oxide surfactant.

2. The antimicrobial composition of claim 1, in which the zinc ions have a concentration from 2.5×10⁻³ to 6.8×10⁻³ mol/L.

3. The antimicrobial composition of claim 1, in which the zinc ions have a concentration from 3.1×10⁻³ to 6.2×10⁻³ mol/L.

4. The antimicrobial composition of claim 1, in which the source of zinc ions is zinc sulfate.

5. The antimicrobial composition of claim 1, in which the cationic active ingredient comprises benzethonium chloride.

6. The antimicrobial composition of claim 1, in which the cationic active ingredient comprises benzalkonium chloride.

7. The antimicrobial composition of claim 1, further comprising 0.5 to 3 wt. % of the composition of cetrimonium chloride.

8. The antimicrobial composition of claim 1, wherein the amine oxide surfactant comprises 0.9 to 1.9 wt. % of the composition of lauryldimethylamine oxide.

9. The antimicrobial composition of claim 1, further comprising a chelating agent, wherein the chelating agent is an organic polyacid.

10. The antimicrobial composition of claim 1, further comprising a non-ionic surfactant, the non-ionic surfactant comprising a polyether with at least 10 ether units.

11. The antimicrobial composition of claim 1, further comprising a cosurfactant, wherein the cosurfactant comprises: cocamidopropyl betaine, sunfloweramidopropyl ethonium sulfate, PEG-120 methyl glucose dioleate, lauryl/myristyl amidopropyl amine oxide, lauramidopropylamine oxide, or combinations thereof.

12. The antimicrobial composition of claim 1, further comprising a dye, a fragrance, a preservative, a pH adjuster, an antiseptic, a humectant, or combinations thereof.

13. The antimicrobial composition of claim 1, wherein the composition achieves an at least 5 log 10 reduction of living E. coli bacteria within 15 seconds of exposure of the bacteria to the composition.

14. An antimicrobial product, comprising:
a container and
an antimicrobial composition housed within the container, wherein the antimicrobial composition comprises:
a quaternary ammonium compound, wherein at least one substitution on the amine comprises a aromatic ring, the quaternary ammonium compound comprising 0.05 to 0.30 wt. % of the composition;
a zinc salt, wherein the zinc ions have a concentration from 1.5×10⁻³ to 7.7×10⁻³ mol/L;
an amine oxide surfactant, comprising 0.5 to 3 wt. % of the antimicrobial composition;
cetrimonium chloride; and
a polar solvent.

15. The product of claim 14, wherein the polar solvent comprises an alcohol with no more than five carbon atoms per molecule.

16. The product of claim 14, wherein the polar solvent comprises water and water comprises no less than 80 wt. % of the antimicrobial composition.

17. The product of claim 14, in which the antimicrobial composition produces at least 4 log 10 reductions in a Gram-negative bacteria population over a 30 second exposure.

18. The product of claim 14, in which the antimicrobial composition further comprises a thickening agent.

19. The product of claim 14, in which the quaternary ammonium compound comprises benzethonium chloride.

20. A method of selectively killing Gram-positive bacteria, the method comprising:
exposing bacteria to a formulation comprising:
0.05 to 0.3 wt. % of benzethonium chloride and/or benzalkonium chloride;
a source of zinc ions, wherein the zinc ions have a concentration from 6.8×10⁻³ to 2×10⁻² mol/L; and
an amine oxide surfactant.

21. The method of claim 20, further comprising rinsing the formulation from the bacteria after exposure.

22. The method of claim 20, wherein the amine oxide surfactant comprises 0.5 wt. % to 3 wt. % of the formulation.
